(19) United States
(12) Patent Application Publication
(10) Pub. No.: US 2025/0197916 A1
(43) Pub. Date: Jun. 19, 2025

(54) METHODS AND COMPOSITIONS FOR SUPPRESSION OF BIOGENIC AMINES PRODUCED BY THE GUT MICROBIOME

(71) Applicant: Cedars-Sinai Medical Center, Los Angeles, CA (US)

(72) Inventors: Ali Rezaie, Beverly Hills, CA (US); Mark Pimentel, Los Angeles, CA (US); Gabriela Leite, Porter Ranch, CA (US); Janice Oh, Los Angeles, CA (US); Yin Chan, Venice, CA (US)

(73) Assignee: Cedars-Sinai Medical Center, Los Angeles, CA (US)

(21) Appl. No.: 18/845,311

(22) PCT Filed: Mar. 20, 2023
(86) PCT No.: PCT/US2023/015663

§ 371 Date: Sep. 9, 2024

Related U.S. Application Data
(60) Provisional application No. 63/322,066, filed on Mar. 21, 2022.

(51) Int. Cl. C12Q 1/06; G01N 33/68

(52) U.S. Cl. CPC C12Q 1/06; G01N 33/6851; G01N 2333/195

(57) ABSTRACT

The present invention provides for methods of treating biogenic amine related conditions. Further provided are methods of detecting biogenic amine related conditions, and selection of treatment for patients in need thereof.

### CROSS-REFERENCE TO RELATED APPLICATIONS

This application includes a claim of priority under 35 U.S.C. § 119(e) to U.S. provisional patent application No. 63/322,066, filed Mar. 21, 2022, the entirety of which is hereby incorporated by reference.

### FIELD OF INVENTION

This invention relates to methods and compositions for suppression of biogenic amines produced by the gut microbiome, and the treatment of human diseases.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

The following embodiments and aspects thereof are described and illustrated in conjunction with compositions and methods which are meant to be exemplary and illustrative, not limiting in scope.

Various embodiments provide for a method of treating a biogenic amine related condition in a subject in need thereof, reducing one or more symptoms in a subject having a biogenic amine related condition, or decreasing biogenic amine level in a subject having a biogenic amine related condition, comprising administering to the subject a therapy selected from: an elemental diet, an enzyme to decrease production of an amine, an enzyme to degrade an amine, an antibiotic to decrease histamine producing microorganisms, putrescine producing microorganisms, cadaverine producing microorganisms, spermine producing microorganisms, or spermidine producing microorganisms in the gut, a probiotic to decrease histamine producing microorganisms, putrescine producing microorganisms, cadaverine producing microorganisms, spermine producing microorganisms, or spermidine producing microorganisms in the gut, or combinations thereof.

In various embodiments, wherein the therapy can be an antibiotic to decrease histamine producing microorganisms, putrescine producing microorganisms, or cadaverine producing microorganisms in the gut. In various embodiments, wherein the therapy can be an antibiotic to decrease spermine producing microorganisms in the gut, or spermidine producing microorganisms in the gut.

In various embodiments, wherein the therapy can be an elemental diet comprising low or absent precursor amino acids for microorganisms to produce histamine, putrescine, cadaverine, spermine or spermidine. In various embodiments, wherein the therapy can be an elemental diet comprising low or absent precursor amino acids for microorganisms to produce histamine, putrescine, or cadaverine. In various embodiments, wherein the therapy can be an elemental diet comprising low or absent precursor amino acids for microorganisms to produce spermine or spermidine. In various embodiments, the precursor amino acid can be histidine, arginine, lysine, ornithine, or combinations thereof. In various embodiments, the therapy can be an elemental diet and the elemental diet is selected from a semi or non-histidine diet, a semi or non-arginine diet, a semi or non-lysine/ornithine diet, or combinations thereof.

In various embodiments, the enzyme to decrease production of an amine can comprise an amino acid decarboxylase to decrease production of histamine, putrescine, or cadaverine.

In various embodiments, the enzyme to decrease production of an amine can comprise an amino acid decarboxylase to decrease production of spermidine or spermine. In various embodiments, the amino acid decarboxylase can be histidine decarboxylase, arginine decarboxylase, or lysine/ornithine decarboxylase. In various embodiments, the amino acid decarboxylase can be spermidine decarboxylase or spermine decarboxylase.

In various embodiments, the enzyme inhibits amino acid decarboxylase and can be selected from the group consisting of alpha-fluoromethylhistidine, alpha-Difluoromethyllysine (DEML), monofluoromethyllysine (MFML), alpha-Difluoromethylarginine, monofluoromethylagmatine, alpha-monofluoromethyl-3-4-dehydroarginine, alpha-Difluoromethylomithine (DFMO), agmatinase inhibitor, arginase inhibitor and combinations thereof.

In various embodiments, the enzyme to degrade an amine can comprise an aminotransferase. In various embodiments, the aminotransferase can be putrescine aminotransferase, cadaverine aminotransferase, or both. In various embodiments, the enzyme to degrade an amine can comprise a monoamine oxidase (MAO), diamine oxidase (DAO), or both. In various embodiments, the enzyme to degrade an amine can be histamine N-methyltransferase (HNMT).

In various embodiments, the enzyme to degrade an amine can be selected from the group consisting of gamma-glutamylcadaverine synthetase, gamma-glutamylputrescine synthetase, gamma-L-glutamylputrescine synthetase, gamma-glutamylcadaverine oxidoreductase, gamma-glutamylputrescine oxidoreductase, or combinations thereof.

In various embodiments, the enzyme to degrade an amine can be selected from the lysine decarboxylase, aminopropylcadverine synthase, S-adenosylmethionine decarboxylase, putrescine aminotransferase, gamma-aminobutyraldehyde dehydrogenase, aminobutyraldehyde dehydrogenase, polyamine aminotransferase, putrescine-pyruvate aminotransferase, aminobutyraldehyde dehydrogenase, 4-aminobutyraldehyde dehydrogenase, glutamate-putrescine ligase, gamma-glutamylputrescine oxidase, gamma-glutamyl-gamma-aminobutraldehyde dehydrogenase, gamma-glutamyl-gamma-aminobutyrate hydrolase, putrescine oxidase, 4-aminobutyradldehyde dehydrogenase, or combinations thereof.

In various embodiments, the enzyme to degrade an amine can comprise diamine oxidase (DAO), and further comprise an enzyme that degrade histamine, putrescine, cadaverine, spermidine, spermine or combinations thereof.

In various embodiments, the enzyme can be enteric coated.

In various embodiments, the antibiotic to decrease histamine producing microorganisms, putrescine producing microorganisms, or cadaverine producing microorganisms in the gut can be an antibiotic effective against gram-negative bacteria. In various embodiments, the antibiotic to decrease spermidine producing microorganisms, or spermine producing microorganisms in the gut can be an antibiotic effective against gram-negative bacteria. In various embodiments, the antibiotic can comprise rifaximin, metronidazole or both. In various embodiments, the antibiotic can be administered in combination with N-acetylcysteine (NAC).

In various embodiments, the probiotic can comprise a histamine metabolizing bacterium, putrascine metabolizing bacterium, cadaverine metabolizing bacterium, spermidine metabolizing bacterium, spermine metabolizing bacterium, or a combination thereof.

In various embodiments, the therapy can further comprise an amine-negative or amine oxidizing bacteria, sodium sorbate, citric acid, succinic acid, malic acid, D-sorbitol, thymol, lemongrass, turmeric, capsaicin, piperine or combinations thereof.

In various embodiments, the biogenic amine related condition can be functional dyspepsia, irritable bowel syndrome (IBS), asthma, migraine, food allergies, environmental allergies, urticaria, mast cell activation syndrome, hereditary alpha tryptasemia, halitosis, or combinations thereof.

In various embodiments, the subject has been detected to have a quantity of one or more of the following microorganisms higher than each microorganism's reference value: Raoultella ornithinolytica, Morganella morganii, Entercoccus faecalis, Proteus mirabilis, Pseudomonas spp., Klebsiella oxytoca, K. aerogenes, K. pneumoniae, Staphylococcus aureus, Stenotrophomonas maltophilia, Escherichia coli, or Enterobacter cloacae, Campylobacter jejuni, Streptococcus pneumoniae, Bacillus subtilis, Archaea, Actinobacteria spp., Firmicutes spp., Proteobacteria spp., or Bacteroidetes spp. In various embodiments, the subject has been detected to have a quantity of one or more of the following microorganisms higher than each microorganism's reference value: Raoultella ornithinolytica, Morganella morganii, Entercoccus faecalis, Proteus mirabilis, Pseudomonas spp., Klebsiella oxytoca, K aerogenes, K. pneumoniae, Staphylococcus aureus, Stenotrophomonas maltophilia, Escherichia coli, or Enterobacter cloacae. In various embodiments, the subject has been detected to have a quantity of one or more of the following microorganisms higher than each microorganism's reference value: Campylobacter jejuni, Streptococcus pneumoniae, Bacillus subtilis, Archaea, S. maltophilia, E. coli, Pseudomonas spp., Actinobacteria spp., Firmicutes spp., Proteobacteria spp., or Bacteroidetes spp.

Various embodiments provide for a method of selecting a therapy for a biogenic amine related condition for a subject, comprising: detecting a quantity of one or more of the following microorganisms: Raoultella ornithinolytica, Morganella morganii, Entercoccus faecalis, Proteus mirabilis, Pseudomonas spp., Klebsiella oxytoca, K. aerogenes, K. pneumoniae, Staphylococcus aureus, Stenotrophomonas maltophilia, Escherichia coli, or Enterobacter cloacae, Campylobacter jejuni, Streptococcus pneumoniae, Bacillus subtilis, Archaea, Actinobacteria spp., Firmicutes spp., Proteobacteria spp., or Bacteroidetes spp.; and selecting a therapy for the subject when the quantity of one or more of the microorganisms are each higher than its reference value, wherein the therapy is selected from: an elemental diet, an enzyme to decrease production of an amine, an enzyme to degrade an amine, an antibiotic to decrease histamine producing microorganisms, putrescine producing microorganisms, cadaverine producing microorganisms, spermidine producing microorganisms, or spermine producing microorganisms in the gut, a probiotic to decrease histamine producing microorganisms, putrescine producing microorganisms, cadaverine producing microorganisms, spermidine producing microorganisms, or spermine producing microorganisms in the gut, or combinations thereof.

In various embodiments, detecting can comprise detecting a quantity of one or more of the following microorganisms: Raoultella ornithinolytica, Morganella morganii, Entercoccus faecalis, Proteus mirabilis, Pseudomonas spp., Klebsiella oxytoca, K. aerogenes, K. pneumoniae, Staphylococcus aureus, Stenotrophomonas maltophilia, Escherichia coli, or Enterobacter cloacae. In various embodiments, detecting can comprise detecting a quantity of one or more of the following microorganisms: detecting a quantity of one or more of the following microorganisms: Campylobacter jejuni, Streptococcus pneumoniae, Bacillus subtilis, Archaea, S. maltophilia, E. coli, Pseudomonas spp., Actinobacteria spp., Firmicutes spp., Proteobacteria spp., or Bacteroidetes spp.

In various embodiments, the detection can be made in a biological sample obtained from the subject. In various embodiments, the biological sample can comprise small bowel aspirate, gut mucosal biopsies, blood, breath, or a combination thereof.

In various embodiments, the method can further comprise administering the selected therapy.

Various embodiments provide for a method of quantifying a microorganism in a biological sample from a subject comprising: assaying the biological sample for one or more of the following microorganisms: Raoultella ornithinolytica, Morganella morganii, Entercoccus faecalis, Proteus mirabilis, Pseudomonas spp., Klebsiella oxytoca, K. aerogenes, K. pneumoniae, Staphylococcus aureus, Stenotrophomonas maltophilia, Escherichia coli, or Enterobacter cloacae, Campylobacter jejuni, Streptococcus pneumoniae, Bacillus subtilis, Archaea, Actinobacteria spp., Firmicutes spp., Proteobacteria spp., or Bacteroidetes spp.; and detecting the quantity of the one or more microorganisms, wherein the subject has a biogenic amine related condition or exhibit a symptom of the biogenic amine related condition.

In various embodiments, assaying can comprise assaying the biological sample for one or more of the following microorganisms: Raoultella ornithinolytica, Morganella morganii, Entercoccus faecalis, Proteus mirabilis, Pseudomonas spp., Klebsiella oxytoca, K. aerogenes, K. pneumoniae, Staphylococcus aureus, Stenotrophomonas maltophilia, Escherichia coli, or Enterobacter cloacae.

In various embodiments, assaying can comprise assaying the biological sample for one or more of the following microorganisms: Campylobacter jejuni, Streptococcus pneumoniae, Bacillus subtilis, Archaea, S. maltophilia, E. coli, Pseudomonas spp., Actinobacteria spp., Firmicutes spp., Proteobacteria spp., or Bacteroidetes spp.

In various embodiments, the method can further comprise comparing the quantity of the one or more microorganisms to each microorganism's reference value.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIGS. 1A and 1B depict Venn diagrams of enteric bacterial species with biogenic amine-producing enzymes based on current literature. Enzyme activity in each bacteria species leads to independent production of histamine, cadaverine, or putrescine, as well as spermidine and spermine.

FIG. 2 shows the results of the analysis of bacterial species in 102 aspirate cultures taken from endoscopic small bowel aspiration from IBS patients with refractory symptoms. Aspirate cultures in study sample grew at least one biogenic amine producing bacteria. Data presented as percentage, as well as female proportion and age (mean±1 SD). Bar graph of most common bacterial species found in aspirate cultures, presented as percentages of total.

FIG. 3A shows that putrescine, cadaverine and histamine are detectable in the lumen of the small bowel.

FIG. 3B shows the descriptive analysis (small bowel luminal concentration—ng/mL).

FIG. 4 shows that putrescine and cadaverine small bowel luminal concentrations are higher than histamine.

FIG. 5A shows that Subjects with SIBO have higher biogenic amines concentrations in the small bowel lumen when compared to non-SIBO.

FIG. 5B shows the descriptive analysis (small bowel luminal concentration in ng/mL).

FIG. 6 shows that small bowel luminal levels of putrescine and cadaverine correlates with GI symptoms severity.

FIG. 7A shows the aspirate level of putrescine vs. aspirate methyl-histamine (center) and aspirate level of cadaverine vs. aspirate methyl-histamine (bottom).

FIG. 7B shows the aspirate level of putrescine vs. biopsy methyl-histamine (top) and aspirate level of cadaverine vs. biopsy methyl-histamine (bottom).

FIG. 8A-8E shows the degradation pathways of amines. (Pathways are prior art.)

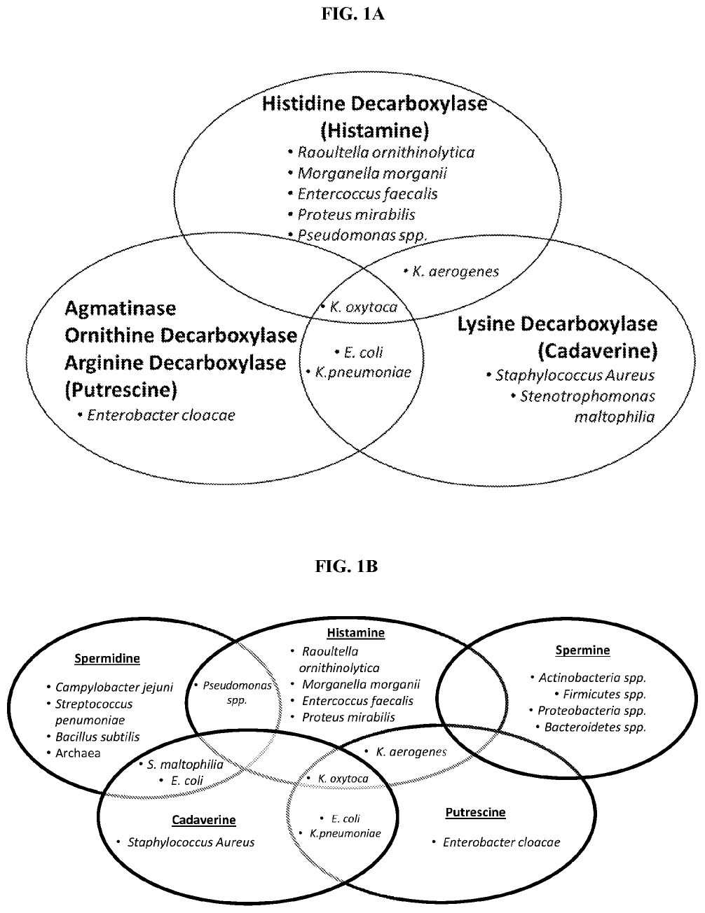

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used herein the term “about” when used in connection with a referenced numeric indication means the referenced numeric indication plus or minus up to 5% of that referenced numeric indication, unless otherwise specifically provided for herein. For example, the language “about 50%” covers the range of 45% to 55%. In various embodiments, the term “about” when used in connection with a referenced numeric indication can mean the referenced numeric indication plus or minus up to 4%, 3%, 2%, 1%, 0.5%, or 0.25% of that referenced numeric indication, if specifically provided for in the claims.

“Treatment” and “treating,” as used herein refer to both therapeutic treatment and prophylactic or preventative measures, wherein the object is to prevent, slow down and/or lessen the disease even if the treatment is ultimately unsuccessful.

As used herein, the term “administering,” refers to the placement an agent as disclosed herein into a subject by a method or route which results in at least partial localization of the agents at a desired site. “Route of administration” may refer to any administration pathway known in the art, including but not limited to aerosol, nasal, via inhalation, oral, anal, intra-anal, peri-anal, transmucosal, transdermal, parenteral, enteral, topical or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions may be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions may be in the form of aerosol, lotion, cream, gel, ointment, suspensions, solutions or emulsions. In accordance with the present disclosure, “administering” may be self-administering. For example, it is considered as “administering” that a subject consumes a composition as disclosed herein.

The term “biological sample” as used herein denotes a sample taken or isolated from a biological organism. Exemplary biological samples include, but are not limited to, cheek swab; mucus; whole blood, blood, serum; plasma; urine; saliva; semen; lymph; fecal extract; intestinal aspirate; sputum; other body fluid or biofluid; cell sample; and tissue sample etc. The term also includes a mixture of the above-mentioned samples. The term “sample” also includes untreated or pretreated (or pre-processed) biological samples.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal may be a human, non-human primate, mouse, rat, dog, cat, horse, or cow, but are not limited to these examples. In addition, the methods described herein may be used to treat domesticated animals and/or pets. In some embodiments, the subject is a human.

“Probiotics” as used herein refers to beneficial bacteria or yeast. Particular examples include but are not limited to bacteria or yeast that degrade biogenic amines, such as histamine metabolizing bacterium, putrascine metabolizing bacterium, cadaverine metabolizing bacterium, spermidine metabolizing bacterium, and spermine metabolizing bacterium.

Histamine is implicated in various diseases. However, the focus has been the endogenous histamine. Described herein, we show that continuous exogenous histamine production from gut microbiome can occur in the gut, and reducing exogenous histamine production, reducing inhibition of histamine catabolism, or both can improve and/or cure these diseases. Also, detection of cadaverine and putrescine in the gut lumen, gut mucosal biopsies can be used to detect the presence of putrescine and cadaverine-producing bacteria in the gut, and diagnose diseases that can be treated by reducing the presence of putrescine and cadaverine-producing bacteria.

Histamine has been implicated in various diseases including functional dyspepsia, IBS, asthma, migraine, food allergies, environmental allergies, urticarial, Hereditary alpha tryptasemia, and mast cell activation syndrome (MCAS). Cadaverine has been implicated in halitosis.

Several bacterial species can independently produce histamine by converting histidine to histamine using the pyridoxal phosphate-dependent enzyme, histidine decarboxylase. One example is Raoultella ornithinolytica, a Gram-negative, aerobic, non-motile rod, that is commonly found in fish, water, and soil. In humans, Raoultella species can cause illnesses including enteric fevers and the scombroid syndrome, which precipitates acute symptoms of vomiting, flushing, abdominal pain and diarrhea due to high histamine levels in consumed fish. Histidine is converted by bacteria to histamine in improperly stored fish, and following consumption, is absorbed in the GI tract, triggering various symptoms. Histamine is not the only biogenic amine involved in scombroid poisoning. Cadaverine, a diamine compound mainly produced by Stenotrophomonas maltophilia an aerobic, non-fermentative, Gram-negative bacterium, has also been shown to facilitate the process by suppression of the histamine metabolizing enzyme-histamine N-methyl transferase and diamine oxidase and the histamine binding to mucin. Detection of known histamine-producing bacteria along with cadaverine and putrescine-producing bacteria in the human gut as described herein opens up the possibility of a mast-cell independent microbiome-dependent pathway for histamine-induced disease pathways including visceral hypersensitivity and new therapeutic modalities.

We performed endoscopic small bowel aspiration and culture in IBS patients with refractory symptoms to assess for small intestinal bacterial overgrowth. Of 102 (66.7% female, mean age 49.7±12.9 years) patients with functional symptoms who underwent small bowel aspiration during esophagogastroduodenoscopy from June 2015 to June 2021, 9 (8.8%; 44.4% female, mean age 59.7±12.6) grew R. ornithinolytica while S. maltophilia, grew in 8 (7.8%; 87.5% female, mean age 52.4±14.1). Three subjects simultaneously had both S. maltophilia and R. ornithinolytica, a statistically significant overlap (P=0.02), suggesting a potential syntrophic relationship between these two species. The presence of these R. ornithinolytica and S. maltophilia confirms that histamine and cadaverine producing bacteria can colonize the small bowel.

Various embodiments of the present invention utilize suppression of cadaverine, putrescine and histamine produced by gut microbiome in the lumen to decrease the harmful effects of histamine. Histamine dependent disease are treated with anti-histamine, anti-cadaverine, anti-putrescine or any combination of these therapies in accordance to various embodiments of the present invention. Exogenous histamine can be suppressed in various ways which are completely different from endogenous histamine. Further, the therapies described herein can be given in conjunction with a mucolytic such as NAC to decrease biofilm and increase intraluminal penetrance of these interventions

Various embodiments of the present invention provide for a method of treating a biogenic amine related condition in a subject in need thereof, comprising administering a therapy selected from: an elemental diet, an enzyme to decrease production of an amine, an enzyme to degrade an amine, an antibiotic to decrease histamine producing microorganisms, putrescine producing microorganisms, cadaverine producing microorganisms in the gut, spermine producing microorganisms in the gut, or spermidine producing microorganisms in the gut, a probiotic to decrease histamine producing microorganisms, putrescine producing microorganisms, cadaverine producing microorganisms in the gut, spermine producing microorganisms in the gut, or spermidine producing microorganisms in the gut, or combinations thereof.

Various embodiments of the present invention provide for a method of treating a biogenic amine related condition in a subject in need thereof, comprising administering a therapy selected from: an elemental diet, an enzyme to decrease production of an amine, an enzyme to degrade an amine, an antibiotic to decrease histamine producing microorganisms, putrescine producing microorganisms, or cadaverine producing microorganisms in the gut, a probiotic to decrease histamine producing microorganisms, putrescine producing microorganisms, or cadaverine producing microorganisms in the gut, or combinations thereof.

Various embodiments of the present invention provide for a method of treating a biogenic amine related condition in a subject in need thereof, comprising administering a therapy selected from: an elemental diet, an enzyme to decrease production of an amine, an enzyme to degrade an amine, an antibiotic to decrease spermine producing microorganisms in the gut, or spermidine producing microorganisms in the gut, a probiotic to decrease spermine producing microorganisms in the gut, or spermidine producing microorganisms in the gut, or combinations thereof.

Various embodiments of the present invention provide for a method of reducing one or more symptoms in a subject having a biogenic amine related condition, comprising administering a therapy selected from: an elemental diet, an enzyme to decrease production of an amine, an enzyme to degrade an amine, an antibiotic to decrease histamine producing microorganisms, putrescine producing microorganisms, cadaverine producing microorganisms in the gut, spermine producing microorganisms in the gut, or spermidine producing microorganisms in the gut, a probiotic to decrease histamine producing microorganisms, putrescine producing microorganisms, cadaverine producing microorganisms in the gut, spermine producing microorganisms in the gut, or spermidine producing microorganisms in the gut, or combinations thereof.

Various embodiments of the present invention provide for a method of reducing one or more symptoms in a subject having a biogenic amine related condition, comprising administering a therapy selected from: an elemental diet, an enzyme to decrease production of an amine, an enzyme to degrade an amine, an antibiotic to decrease histamine producing microorganisms, putrescine producing microorganisms, or cadaverine producing microorganisms in the gut, a probiotic to decrease histamine producing microorganisms, putrescine producing microorganisms, or cadaverine producing microorganisms in the gut, or combinations thereof.

Various embodiments of the present invention provide for a method of reducing one or more symptoms in a subject having a biogenic amine related condition, comprising administering a therapy selected from: an elemental diet, an enzyme to decrease production of an amine, an enzyme to degrade an amine, an antibiotic to decrease spermine producing microorganisms in the gut, or spermidine producing microorganisms in the gut, a probiotic to decrease spermine producing microorganisms in the gut, or spermidine producing microorganisms in the gut, or combinations thereof.

Accordingly, the one or more symptoms in the subject is decreased as compared to before the administration of the therapy. Examples of symptoms include but are not limited to bloating, abdominal pain, excess gas, diarrhea, regurgitation, reflux, non-cardiac chest pain, nausea, vomiting, flushing, allergic reactions, food sensitivities, urticaria, and brain fog.

Various embodiments of the present invention provide for a method of decreasing biogenic amine level in a subject having a biogenic amine related condition, comprising administering a therapy selected from: an elemental diet, an enzyme to decrease production of an amine, an enzyme to degrade an amine, an antibiotic to decrease histamine producing microorganisms, putrescine producing microorganisms, cadaverine producing microorganisms in the gut, spermine producing microorganisms in the gut, or spermidine producing microorganisms in the gut, a probiotic to decrease histamine producing microorganisms, putrescine producing microorganisms, cadaverine producing microorganisms in the gut, spermine producing microorganisms in the gut, or spermidine producing microorganisms in the gut, or combinations thereof.

Various embodiments of the present invention provide for a method of decreasing biogenic amine level in a subject having a biogenic amine related condition, comprising administering a therapy selected from: an elemental diet, an enzyme to decrease production of an amine, an enzyme to degrade an amine, an antibiotic to decrease histamine producing microorganisms, putrescine producing microorganisms, or cadaverine producing microorganisms in the gut, a probiotic to decrease histamine producing microorganisms, putrescine producing microorganisms, or cadaverine producing microorganisms in the gut, or combinations thereof.

Various embodiments of the present invention provide for a method of decreasing biogenic amine level in a subject having a biogenic amine related condition, comprising administering a therapy selected from: an elemental diet, an enzyme to decrease production of an amine, an enzyme to degrade an amine, an antibiotic to decrease spermine producing microorganisms in the gut, or spermidine producing microorganisms in the gut, a probiotic to decrease spermine producing microorganisms in the gut, or spermidine producing microorganisms in the gut, or combinations thereof.

Accordingly, the biogenic amine level in the subject is decreased; for example, in the gut lumen, as compared to before the administration of the therapy. Additional locations that the biogenic amine level is decreased include but are not limited to blood and tissue, and biofilm on or in a subject's body.

In various embodiments, the therapy is an elemental diet. In various embodiments, the therapy is an enzyme to decrease production of an amine. In various embodiments, the therapy is an enzyme to degrade an amine. In various embodiments, the therapy is an antibiotic to decrease histamine producing microorganisms, putrescine producing microorganisms, or cadaverine producing microorganisms in the gut. In various embodiments, the therapy is a probiotic to decrease histamine producing microorganisms, putrescine producing microorganisms, or cadaverine producing microorganisms in the gut. In various embodiments, the therapy is a combination of an elemental diet, an enzyme to decrease production of an amine, an enzyme to degrade an amine, an antibiotic to decrease histamine producing microorganisms, putrescine producing microorganisms, or cadaverine producing microorganisms in the gut, a probiotic to decrease histamine producing microorganisms, putrescine producing microorganisms, or cadaverine producing microorganisms in the gut.

In various embodiments, the therapy is an elemental diet comprising low or absent precursor amino acids for microorganisms to produce histamine, putrescine, cadaverine, spermine or spermidine. In various embodiments, the therapy is an elemental diet comprising low or absent precursor amino acids for microorganisms to produce histamine, putrescine, or cadaverine. In various embodiments, the therapy is an elemental diet comprising low or absent precursor amino acids for microorganisms to produce spermine or spermidine. In various embodiments, the precursor amino acid is histidine, arginine, lysine, ornithine, or combinations thereof. In various embodiments, the therapy is an elemental diet and the elemental diet is selected from a semi or non-histidine diet, a semi or non-arginine diet, a semi or non-lysine/ornithine diet, or combinations thereof.

While low histamine diets exist, but the embodiments described herein include a diet that is low or absent precursor amino acids for bacteria to produce histamine, putrescine, cadaverine (i.e., histidine, arginine, lysine/ornithine respectively). This decreases both production of histamine and production of putrescine and cadaverine, which decrease degradation of histamine.

In various embodiments, the enzyme to decrease production of an amine comprises an amino acid decarboxylase to decrease production of histamine, putrescine, or cadaverine. In various embodiments, the enzyme to decrease production of an amine comprises an amino acid decarboxylase to decrease production of spermidine or spermine.

In various embodiments, the amino acid decarboxylase is histidine decarboxylase, arginine decarboxylase, or lysine/ornithine decarboxylase. In various embodiments, the amino acid decarboxylase is spermidine decarboxylase or spermine decarboxylase.

In various embodiments, the enzyme inhibits amino acid decarboxylase and is selected from the group consisting of alpha-fluoromethylhistidine, alpha-Difluoromethyllysine (DEML), monofluoromethyllysine (MFML), alpha-Difluoromethylarginine, monofluoromethylagmatine, alpha-monofluoromethyl-3-4-dehydroarginine, alpha-Difluoromethylomithine (DFMO), agmatinase inhibitor, arginase inhibitor and combinations thereof.

In various embodiments, the enzyme inhibits amino acid decarboxylase and is a proteolytic example; for example trypsin or chymotrypsin.

In various embodiments, the enzyme to degrade an amine comprises an aminotransferase. In various embodiments, the amino transferase is putrescine aminotransferase, cadaverine aminotransferase, or both. In various embodiments, the amino transferase is spermidine aminotransferase, spermine aminotransferase, or both.

In various embodiments, the enzyme to degrade an amine comprises a monoamine oxidase (MAO), diamine oxidase (DAO), or both. In various embodiments, the enzyme to degrade an amine is histamine N-methyltransferase (HNMT).

In various embodiments, the enzyme to degrade an amine is selected from the group consisting of gamma-glutamylcadaverine synthetase, gamma-glutamylputrescine synthetase, gamma-L-glutamylputrescine synthetase, gamma-glutamylcadaverine oxidoreductase, gamma-glutamylputrescine oxidoreductase, or combinations thereof.

In various embodiments, the enzyme to degrade an amine is selected from the lysine decarboxylase, aminopropylcadverine synthase, S-adenosylmethionine decarboxylase, putrescine aminotransferase, gamma-aminobutyraldehyde dehydrogenase, aminobutyraldehyde dehydrogenase, polyamine aminotransferase, putrescine-pyruvate aminotransferase, aminobutyraldehyde dehydrogenase, 4-aminobutyraldehyde dehydrogenase, glutamate-putrescine ligase, gamma-glutamylputrescine oxidase, gamma-glutamyl-gamma-aminobutraldehyde dehydrogenase, gamma-glutamyl-gamma-aminobutyrate hydrolase, putrescine oxidase, 4-aminobutyradldehyde dehydrogenase, or combinations thereof. Pathways associated with metabolism of polyamines, including cadaverine and cadaverine bioproducts (aminopropylcadaverine and siderophores—desferrioxamine B/E, and bisucaberin), were also enriched in SIBO subjects and associated with increased severity of self-reported GI symptoms. Accordingly, utilizing one or more of these enzymes to degrade the amine (biogenic amine) is beneficial to subjects having one or more symptoms of biogenic amine condition to alleviate their symptom(s) or lower the level of biogenic amines.

In various embodiments, the enzyme to degrade an amine comprises diamine oxidase (DAO), and further comprise an enzyme that degrade putrescine, cadaverine, or both.

In various embodiments, the enzyme to degrade an amine comprises diamine oxidase (DAO), and further comprise an enzyme that degrade histamine, putrescine, cadaverine, spermine, spermidine or combinations thereof.

In various embodiments, the enzyme to degrade an amine comprises diamine oxidase (DAO), and further comprises gamma-glutamylcadaverine synthetase, gamma-glutamylputrescine synthetase, gamma-L-glutamylputrescine synthetase, gamma-glutamylcadaverine oxidoreductase, gamma-glutamylputrescine oxidoreductase, or combinations thereof.

In various embodiments, the enzyme to degrade an amine comprises diamine oxidase (DAO), and further comprises lysine decarboxylase, aminopropylcadverine synthase, S-adenosylmethionine decarboxylase, putrescine aminotransferase, gamma-aminobutyraldehyde dehydrogenase, aminobutyraldehyde dehydrogenase, polyamine aminotransferase, putrescine-pyruvate aminotransferase, aminobutyraldehyde dehydrogenase, 4-aminobutyraldehyde dehydrogenase, glutamate-putrescine ligase, gamma-glutamylputrescine oxidase, gamma-glutamyl-gamma-aminobutraldehyde dehydrogenase, gamma-glutamyl-gamma-aminobutyrate hydrolase, putrescine oxidase, 4-aminobutyradldehyde dehydrogenase, or combinations thereof.

In various embodiments, the enzyme is enteric coated.

In various embodiments, the therapy comprises an antibiotic to decrease histamine producing microorganisms, putrescine producing microorganisms, or cadaverine producing microorganisms in the gut. In various embodiments, the therapy comprises an antibiotic to decrease spermine producing microorganisms in the gut, or spermidine producing microorganisms in the gut.

In various embodiments, the antibiotic to decrease histamine producing microorganisms or putrescine producing microorganisms in the gut is an antibiotic effective against gram-negative bacteria. In various embodiments, the antibiotic to decrease histamine producing microorganisms, putrescine producing microorganisms, or cadaverine producing microorganisms in the gut comprises rifaximin, metronidazole or both. In various embodiments, the antibiotic to decrease cadaverine producing microorganisms in the gut is an antibiotic effective against gram-negative bacteria. In various embodiments, the antibiotic to decrease spermine producing microorganisms in the gut, or spermidine producing microorganisms in the gut is an antibiotic effective against gram-negative bacteria.

In various embodiments, the antibiotic is administered in combination with N-acetylcysteine (NAC).

In various embodiments, the probiotic comprises histamine metabolizing bacterium, putrascine metabolizing bacterium, cadaverine metabolizing bacterium, spermidine metabolizing bacterium, or spermine metabolizing bacterium.

In various embodiments, the probiotic comprises Histaminolytic bacteria, Virgibacillus sp., Natrinema gari, Brevibacterium linen, Arthrobacter crystallopoietes, Staphylococcus xylosus, Yarrowia lipolytica or combinations thereof.

In various embodiments, the therapy further comprises amine-negative or amine oxidizing bacteria, sodium sorbate, citric acid, succinic acid, malic acid, D-sorbitol, thymol, lemongrass, turmeric, capsaicin, piperine or combinations thereof.

In various embodiments, the therapy comprises a combination of (1) DAO and (2) an elemental diet, an enzyme to decrease production of an amine, an enzyme to degrade an amine, an antibiotic to decrease histamine producing microorganisms, putrescine producing microorganisms, cadaverine producing microorganisms spermine producing microorganisms or spermidine producing microorganisms in the gut, a probiotic to decrease histamine producing microorganisms, putrescine producing microorganisms, cadaverine producing microorganisms, spermine producing microorganisms, or spermidine producing microorganisms in the gut.

In various embodiments, the biogenic amine related condition is functional dyspepsia, irritable bowel syndrome (IBS), asthma, migraine, food allergies, environmental allergies, urticaria, mast cell activation syndrome, halitosis, hereditary alpha tryptasemia, or combinations thereof.

In various embodiments, the subject has been detected to have a quantity of one or more of the following microorganisms higher than each microorganism's reference value: Raoultella ornithinolytica, Morganella morganii, Entercoccus faecalis, Proteus mirabilis, Pseudomonas spp., Klebsiella oxytoca, K. aerogenes, K. pneumoniae, Staphylococcus aureus, Stenotrophomonas maltophilia, Escherichia coli, or Enterobacter cloacae, Campylobacter jejuni, Streptococcus pneumoniae, Bacillus subtilis, Archaea, Actinobacteria spp., Firmicutes spp., Proteobacteria spp., or Bacteroidetes spp. In various embodiments, the subject has been detected to have a quantity of one or more of the following microorganisms higher than each microorganism's reference value: Raoultella ornithinolytica, Morganella morganii, Entercoccus faecalis, Proteus mirabilis, Pseudomonas spp., Klebsiella oxytoca, K. aerogenes, K. pneumoniae, Staphylococcus aureus, Stenotrophomonas maltophilia, Escherichia coli, or Enterobacter cloacae. In various embodiments, the subject has been detected to have a quantity of one or more of the following microorganisms higher than each microorganism's reference value: Campylobacter jejuni, Streptococcus pneumoniae, Bacillus subtilis, Archaea, S. maltophilia, E. coli, Pseudomonas spp., Actinobacteria spp., Firmicutes spp., Proteobacteria spp., or Bacteroidetes spp.

The reference value can depend on the type of biogenic amine related condition. Different types of biogenic amine related condition may have a different reference values. In some embodiments, the reference value can be established from biological samples from a healthy subject. For example, if the biological sample is stool, then the reference value can be obtained from the stools of a healthy subject; that is, a subject who does not have the biogenic amine related condition. In other embodiments, the reference value is the average microorganism count for the same type of biological sample from a population of healthy subjects. In some embodiments, the population of healthy subjects can range from at least three healthy individuals to 25 healthy individuals, from at least 26 healthy individuals to 50 healthy individuals, from at least 51 healthy individuals to 100 healthy individuals, and even more than 100 healthy individuals.

### Selecting Therapy

Various embodiments provide for a method of selecting a therapy for a biogenic amine related condition for a subject, comprising: detecting a quantity of one or more of the following microorganisms: Raoultella ornithinolytica, Morganella morganii, Entercoccus faecalis, Proteus mirabilis, Pseudomonas spp., Klebsiella oxytoca, K. aerogenes, K. pneumoniae, Staphylococcus aureus, Stenotrophomonas maltophilia, Escherichia coli, or Enterobacter cloacae, Campylobacter jejuni, Streptococcus pneumoniae, Bacillus subtilis, Archaea, Actinobacteria spp., Firmicutes spp., Proteobacteria spp., or Bacteroidetes spp.; and selecting a therapy for the subject when the quantity of one or more of the microorganisms are each higher than its reference value, wherein the therapy is selected from: an elemental diet, an enzyme to decrease production of an amine, an enzyme to degrade an amine, an antibiotic to decrease histamine producing microorganisms, putrescine producing microorganisms, cadaverine producing microorganisms, spermidine producing microorganisms, or spermine producing microorganisms in the gut, a probiotic to decrease histamine producing microorganisms, putrescine producing microorganisms, cadaverine producing microorganisms, spermidine producing microorganisms, or spermine producing microorganisms in the gut, or combinations thereof.

In various embodiments, detecting comprises detecting a quantity of one or more of the following microorganisms: Raoultella ornithinolytica, Morganella morganii, Entercoccus faecalis, Proteus mirabilis, Pseudomonas spp., Klebsiella oxytoca, K. aerogenes, K. pneumoniae, Staphylococcus aureus, Stenotrophomonas maltophilia, Escherichia coli, or Enterobacter cloacae.

In various embodiments, detecting comprises detecting a quantity of one or more of the following microorganisms: detecting a quantity of one or more of the following microorganisms: Campylobacter jejuni, Streptococcus pneumoniae, Bacillus subtilis, Archaea, S. maltophilia, E. coli, Pseudomonas spp., Actinobacteria spp., Firmicutes spp., Proteobacteria spp., or Bacteroidetes spp.

In various embodiments, the detection is made in a biological sample obtained from the subject. In various embodiments, the biological sample comprises small bowel aspirate, gut mucosal biopsies, blood, breath, or a combination thereof.

In various embodiments, the method further comprises administering the selected therapy.

In various embodiments, the therapy is an elemental diet. In various embodiments, the therapy is an enzyme to decrease production of an amine. In various embodiments, the therapy is an enzyme to degrade an amine. In various embodiments, the therapy is an antibiotic to decrease histamine producing microorganisms, putrescine producing microorganisms, or cadaverine producing microorganisms in the gut. In various embodiments, the therapy is a probiotic to decrease histamine producing microorganisms, putrescine producing microorganisms, or cadaverine producing microorganisms in the gut. In various embodiments, the therapy is a combination of an elemental diet, an enzyme to decrease production of an amine, an enzyme to degrade an amine, an antibiotic to decrease histamine producing microorganisms, putrescine producing microorganisms, or cadaverine producing microorganisms in the gut, a probiotic to decrease histamine producing microorganisms, putrescine producing microorganisms, or cadaverine producing microorganisms in the gut.

In various embodiments, the therapy is an elemental diet comprising low or absent precursor amino acids for microorganisms to produce histamine, putrescine, cadaverine, spermine or spermidine. In various embodiments, the therapy is an elemental diet comprising low or absent precursor amino acids for microorganisms to produce histamine, putrescine, or cadaverine. In various embodiments, the therapy is an elemental diet comprising low or absent precursor amino acids for microorganisms to produce spermine or spermidine. In various embodiments, the precursor amino acid is histidine, arginine, lysine, ornithine, or combinations thereof. In various embodiments, the therapy is an elemental diet and the elemental diet is selected from a semi or non-histidine diet, a semi or non-arginine diet, a semi or non-lysine/ornithine diet, or combinations thereof.

Again, while low histamine diets exist, but the embodiments described herein include a diet that is low or absent precursor amino acids for bacteria to produce histamine, putrescine, cadaverine (i.e., histidine, arginine, lysine/orithine respectively). This decreases both production of histamine and production of putrescine and cadaverine, which decrease degradation of histamine.

In various embodiments, the enzyme to decrease production of an amine comprises an amino acid decarboxylase to decrease production of histamine, putrescine, or cadaverine. In various embodiments, the enzyme to decrease production of an amine comprises an amino acid decarboxylase to decrease production of spermidine or spermine.

In various embodiments, the amino acid decarboxylase is histidine decarboxylase, arginine decarboxylase, or lysine/ornithine decarboxylase. In various embodiments, the amino acid decarboxylase is spermidine decarboxylase or spermine decarboxylase.

In various embodiments, the enzyme inhibits amino acid decarboxylase and is selected from the group consisting of alpha-fluoromethylhistidine, alpha-Difluoromethyllysine (DEML), monofluoromethyllysine (MFML), alpha-Difluoromethylarginine, monofluoromethylagmatine, alpha-monofluoromethyl-3-4-dehydroarginine, alpha-Difluoromethylomithine (DFMO), agmatinase inhibitor, arginase inhibitor and combinations thereof.

In various embodiments, the enzyme inhibits amino acid decarboxylase and is a proteolytic example; for example trypsin or chymotrypsin.

In various embodiments, the enzyme to degrade an amine comprises an aminotransferase. In various embodiments, the amino transferase is putrescine aminotransferase, cadaverine aminotransferase, or both. In various embodiments, the amino transferase is spermidine aminotransferase, spermine aminotransferase, or both.

In various embodiments, the enzyme to degrade an amine comprises a monoamine oxidase (MAO), diamine oxidase (DAO), or both. In various embodiments, the enzyme to degrade an amine is histamine N-methyltransferase (HNMT).

In various embodiments, the enzyme to degrade an amine is selected from the group consisting of gamma-glutamylcadaverine synthetase, gamma-glutamylputrescine synthetase, gamma-L-glutamylputrescine synthetase, gamma-glutamylcadaverine oxidoreductase, gamma-glutamylputrescine oxidoreductase, or combinations thereof.

In various embodiments, the enzyme to degrade an amine is selected from the lysine decarboxylase, aminopropylcadverine synthase, S-adenosylmethionine decarboxylase, putrescine aminotransferase, gamma-aminobutyraldehyde dehydrogenase, aminobutyraldehyde dehydrogenase, polyamine aminotransferase, putrescine-pyruvate aminotransferase, aminobutyraldehyde dehydrogenase, 4-aminobutyraldehyde dehydrogenase, glutamate-putrescine ligase, gamma-glutamylputrescine oxidase, gamma-glutamyl-gamma-aminobutraldehyde dehydrogenase, gamma-glutamyl-gamma-aminobutyrate hydrolase, putrescine oxidase, 4-aminobutyradldehyde dehydrogenase, or combinations thereof.

In various embodiments, the enzyme to degrade an amine comprises diamine oxidase (DAO), and further comprise an enzyme that degrade putrescine, cadaverine, or both. In various embodiments, the enzyme to degrade an amine comprises diamine oxidase (DAO), and further comprise an enzyme that degrade histamine, putrescine, cadaverine, spermine, spermidine or combinations thereof.

In various embodiments, the enzyme to degrade an amine comprises diamine oxidase (DAO), and further comprises gamma-glutamylcadaverine synthetase, gamma-glutamylputrescine synthetase, gamma-L-glutamylputrescine synthetase, gamma-glutamylcadaverine oxidoreductase, gamma-glutamylputrescine oxidoreductase, or combinations thereof.

In various embodiments, the enzyme to degrade an amine comprises diamine oxidase (DAO), and further comprises lysine decarboxylase, aminopropylcadverine synthase, S-adenosylmethionine decarboxylase, putrescine aminotransferase, gamma-aminobutyraldehyde dehydrogenase, aminobutyraldehyde dehydrogenase, polyamine aminotransferase, putrescine-pyruvate aminotransferase, aminobutyraldehyde dehydrogenase, 4-aminobutyraldehyde dehydrogenase, glutamate-putrescine ligase, gamma-glutamylputrescine oxidase, gamma-glutamyl-gamma-aminobutraldehyde dehydrogenase, gamma-glutamyl-gamma-aminobutyrate hydrolase, putrescine oxidase, 4-aminobutyradldehyde dehydrogenase, or combinations thereof.

In various embodiments, the enzyme is enteric coated.

In various embodiments, the therapy comprises an antibiotic to decrease histamine producing microorganisms, putrescine producing microorganisms, or cadaverine producing microorganisms in the gut. In various embodiments, the therapy comprises an antibiotic to decrease spermine producing microorganisms in the gut, or spermidine producing microorganisms in the gut.

In various embodiments, the antibiotic to decrease histamine producing microorganisms or putrescine producing microorganisms in the gut is an antibiotic effective against gram-negative bacteria. In various embodiments, the antibiotic to decrease histamine producing microorganisms, putrescine producing microorganisms, or cadaverine producing microorganisms in the gut comprises rifaximin, metronidazole or both. In various embodiments, the antibiotic to decrease cadaverine producing microorganisms in the gut is an antibiotic effective against gram-negative bacteria. In various embodiments, the antibiotic to decrease spermine producing microorganisms in the gut, or spermidine producing microorganisms in the gut is an antibiotic effective against gram-negative bacteria.

In various embodiments, the antibiotic is administered in combination with N-acetylcysteine (NAC).

In various embodiments, the probiotic comprises histamine metabolizing bacterium, putrascine metabolizing bacterium, cadaverine metabolizing bacterium, spermidine metabolizing bacterium, or spermine metabolizing bacterium.

In various embodiments, the probiotic comprises Histaminolytic bacteria, Virgibacillus sp., Natrinema gari, Brevibacterium linen, Arthrobacter crystallopoietes, Staphylococcus xylosus, Yarrowia lipolytica or combinations thereof.

In various embodiments, the therapy further comprises amine-negative or amine oxidizing bacteria, sodium sorbate, citric acid, succinic acid, malic acid, D-sorbitol, thymol, lemongrass, turmeric, capsaicin, piperine or combinations thereof.

In various embodiments, the therapy comprises a combination of (1) DAO and (2) an elemental diet, an enzyme to decrease production of an amine, an enzyme to degrade an amine, an antibiotic to decrease histamine producing microorganisms, putrescine producing microorganisms, cadaverine producing microorganisms spermine producing microorganisms or spermidine producing microorganisms in the gut, a probiotic to decrease histamine producing microorganisms, putrescine producing microorganisms, cadaverine producing microorganisms, spermine producing microorganisms, or spermidine producing microorganisms in the gut.

In various embodiments, the biogenic amine related condition is functional dyspepsia, irritable bowel syndrome (IBS), asthma, migraine, food allergies, environmental allergies, urticaria, mast cell activation syndrome, halitosis, hereditary alpha tryptasemia, or combinations thereof.

In various embodiments, the subject has been detected to have a quantity of one or more of the following microorganisms higher than each microorganism's reference value: Raoultella ornithinolytica, Morganella morganii, Entercoccus faecalis, Proteus mirabilis, Pseudomonas spp., Klebsiella oxytoca, K. aerogenes, K. pneumoniae, Staphylococcus aureus, Stenotrophomonas maltophilia, Escherichia coli, or Enterobacter cloacae, Campylobacter jejuni, Streptococcus pneumoniae, Bacillus subtilis, Archaea, Actinobacteria spp., Firmicutes spp., Proteobacteria spp., or Bacteroidetes spp. In various embodiments, the subject has been detected to have a quantity of one or more of the following microorganisms higher than each microorganism's reference value: Raoultella ornithinolytica, Morganella morganii, Entercoccus faecalis, Proteus mirabilis, Pseudomonas spp., Klebsiella oxytoca, K. aerogenes, K. pneumoniae, Staphylococcus aureus, Stenotrophomonas maltophilia, Escherichia coli, or Enterobacter cloacae. In various embodiments, the subject has been detected to have a quantity of one or more of the following microorganisms higher than each microorganism's reference value: Campylobacter jejuni, Streptococcus pneumoniae, Bacillus subtilis, Archaea, S. maltophilia, E. coli, Pseudomonas spp., Actinobacteria spp., Firmicutes spp., Proteobacteria spp., or Bacteroidetes spp.

The reference value can depend on the type of biogenic amine related condition. Different types of biogenic amine related condition may have a different reference values. In some embodiments, the reference value can be established from biological samples from a healthy subject. For example, if the biological sample is stool, then the reference value can be obtained from the stools of a healthy subject; that is, a subject who does not have the biogenic amine related condition. In other embodiments, the reference value is the average microorganism count for the same type of biological sample from a population of healthy subjects. In some embodiments, the population of healthy subjects can range from at least three healthy individuals to 25 healthy individuals, from at least 26 healthy individuals to 50 healthy individuals, from at least 51 healthy individuals to 100 healthy individuals, and even more than 100 healthy individuals.

### Detection of Presence of Bioamine-Producing Microorganisms

Various embodiments of the present invention provide for methods of detecting the presence of putrescine and cadaverine-producing bacteria in the gut. Detection of cadaverine and putrescine can be done in the gut lumen, via gut mucosal biopsies, via blood and via breath, as examples.

Various embodiments provide for a method of quantifying a microorganism in a biological sample from a subject comprising: assaying the biological sample for one or more of the following microorganisms: Raoultella ornithinolytica, Morganella morganii, Entercoccus faecalis, Proteus mirabilis, Pseudomonas spp., Klebsiella oxytoca, K. aerogenes, K. pneumoniae, Staphylococcus aureus, Stenotrophomonas maltophilia, Escherichia coli, or Enterobacter cloacae, Campylobacter jejuni, Streptococcus pneumoniae, Bacillus subtilis, Archaea, Actinobacteria spp., Firmicutes spp., Proteobacteria spp., or Bacteroidetes spp.; and detecting the quantity of the one or more microorganisms, wherein the subject has a biogenic amine related condition or exhibit a symptom of the biogenic amine related condition.

In various embodiments, assaying comprises assaying the biological sample for one or more of the following microorganisms: Raoultella ornithinolytica, Morganella morganii, Entercoccus faecalis, Proteus mirabilis, Pseudomonas spp., Klebsiella oxytoca, K. aerogenes, K. pneumoniae, Staphylococcus aureus, Stenotrophomonas maltophilia, Escherichia coli, or Enterobacter cloacae.

In various embodiments, assaying comprises assaying the biological sample for one or more of the following microorganisms: Campylobacter jejuni, Streptococcus pneumoniae, Bacillus subtilis, Archaea, S. maltophilia, E. coli, Pseudomonas spp., Actinobacteria spp., Firmicutes spp., Proteobacteria spp., or Bacteroidetes spp.

In various embodiments, the method further comprises comparing the quantity of the one or more microorganisms to each microorganism's reference value.

In various embodiment, the presence of one or more amine producing microorganism above each of their reference level indicates a likely presence of the biogenic amine related condition.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

We have shown in a study of more than 100 patients (102 patients) that (1) biogenic amine-producing bacteria colonize the small bowel of IBS patients; and (2) syntrophic presence of cadaverine/putrescine producing bacteria along with histamine producing bacteria. Cultures were collected from endoscopic small bowel aspiration; statistical significance of the overlap between two culture P-value <0.05.

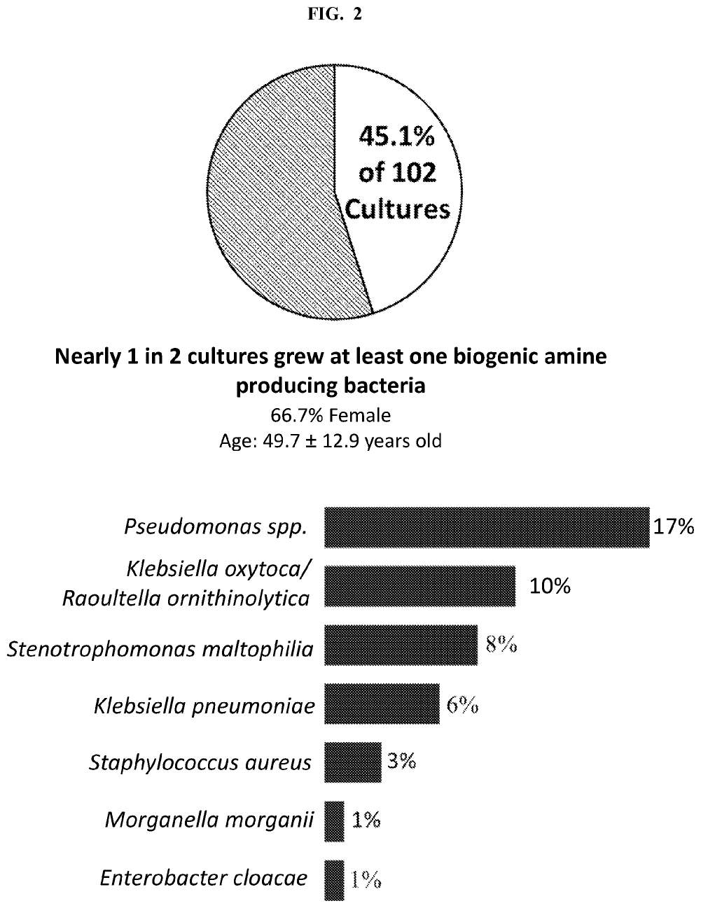

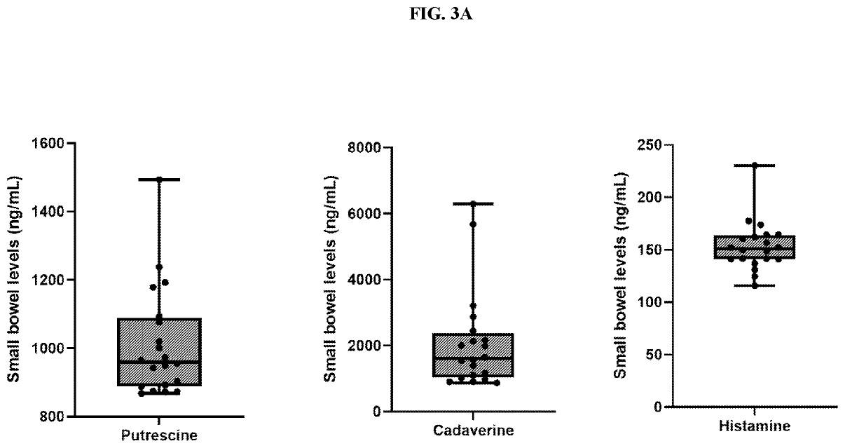

FIG. 2 shows the results of the bacterial species in cultures. The table below shows the results of the overlap of bacterial species.

This is very important as cadaverine/putrescine results in the suppression of DAO enzyme activity and HNMT activity, both of which would have broken down histamine. Hence, the reduction of DAO enzyme activity and HNMT activity, due to the presence of cadaverine/putrescine, increases the histamine levels.

Biogenic amine-producing bacteria colonizing the small bowel of IBS patients. Syntrophic presence of cadaverine/putrescine producing bacteria along with histamine producing bacteria is found. Cadaverine/putrescine results in the suppression of DAO activity, the DAO activity would have broken down histamine; therefore, the reduction of DAO activity increases histamine levels, which causes symptoms of biogenic amine related conditions such as functional dyspepsia, irritable bowel syndrome (IBS), asthma, migraine, food allergies, environmental allergies, urticaria, mast cell activation syndrome, hereditary alpha tryptasemia, and halitosis.

TABLE 1
Percentage of total patients in study sample with overlap of bacterial species in aspirate culture.

| | Pseudomonas | K. oxytoca/R. | S. | K. | S. | M. | E. |
|---|---|---|---|---|---|---|---|
| | spp. | ornithinolytica | maltophilia | pneumoniae | aureus | morganii | cloacae |
| Pseudomonas spp. | — | — | — | — | — | — | — |
| K. oxytoca/ | 6%* | — | — | — | — | — | — |
| R. ornithinolytica | P < 0.01 | | | | | | |
| S. maltophilia | 2% | 4%* | — | — | — | — | — |
| | | P < 0.003 | | | | | |
| K. pneumoniae | 0% | 1% | 1% | — | — | — | — |
| S. aureus | 1% | 0% | 0% | 0% | — | — | — |
| M. morganii | 0% | 0% | 0% | 0% | 0% | — | — |
| E. cloacae | 0% | 0% | 0% | 1% | 0% | 0% | — |

P values indicating statistical significance of overlap of two species. P-value < 0.05 shows significance.

### Example 2

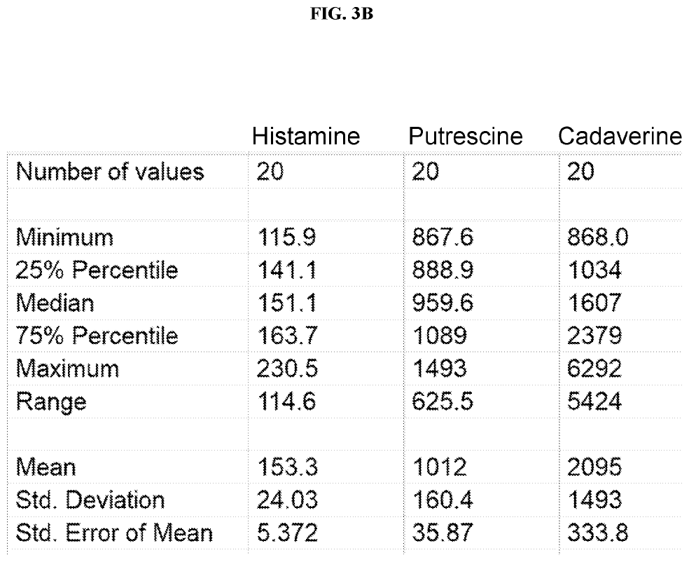

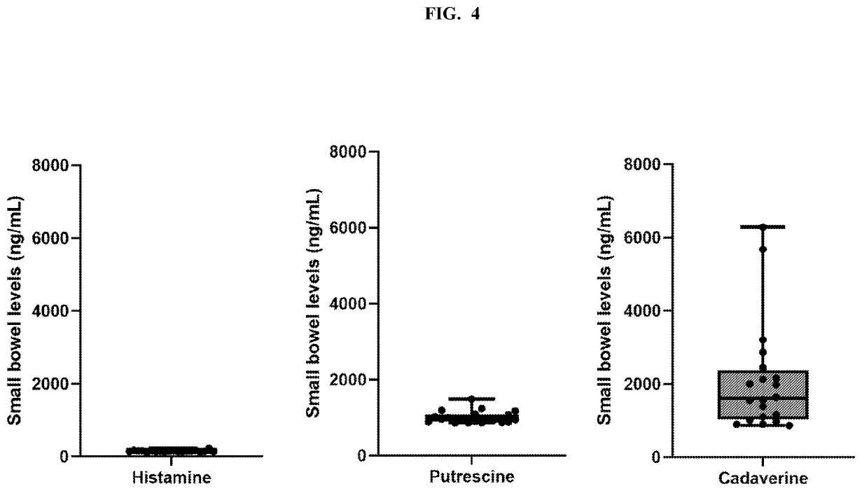

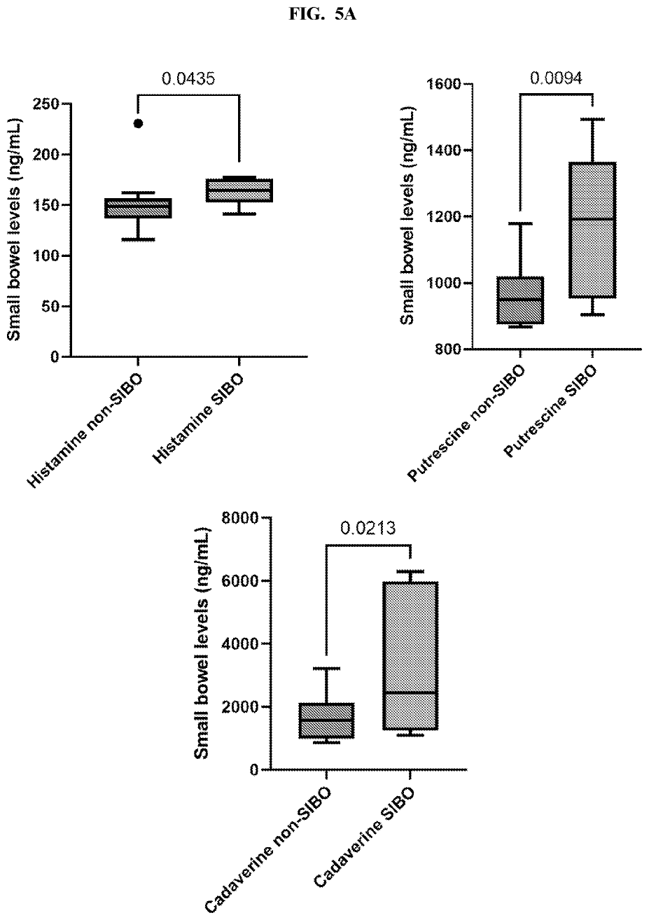

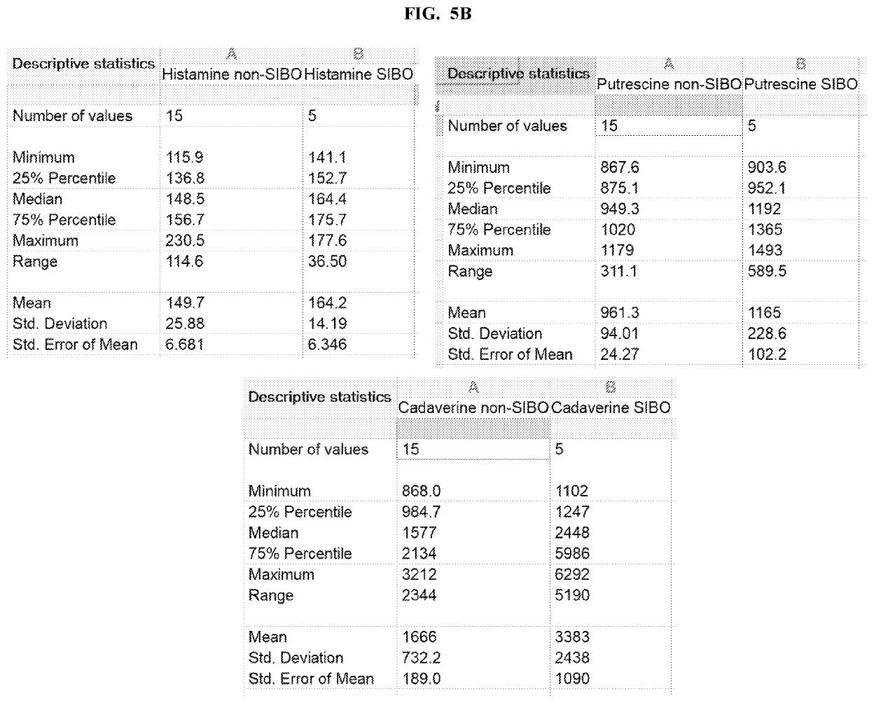

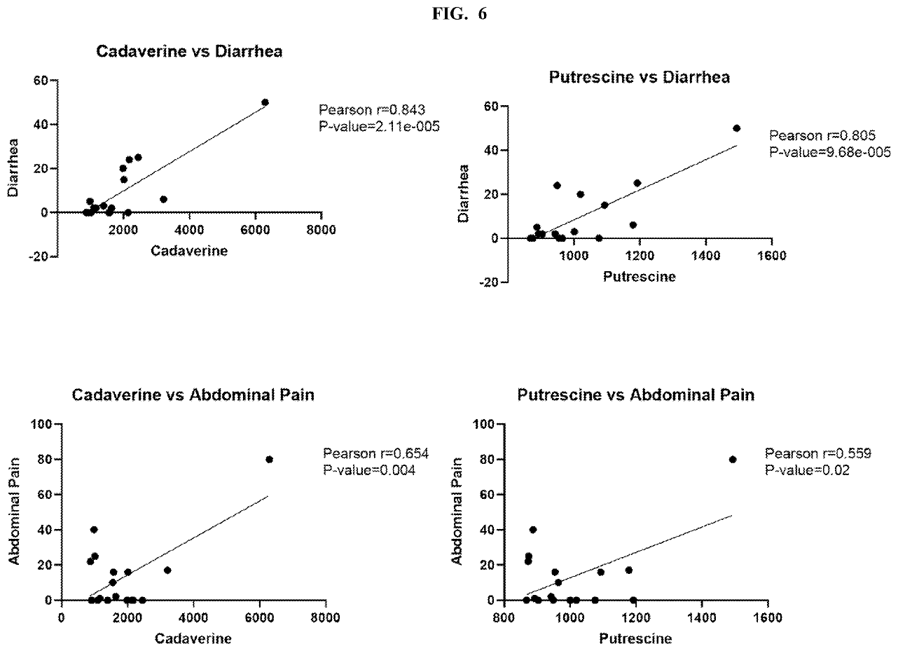

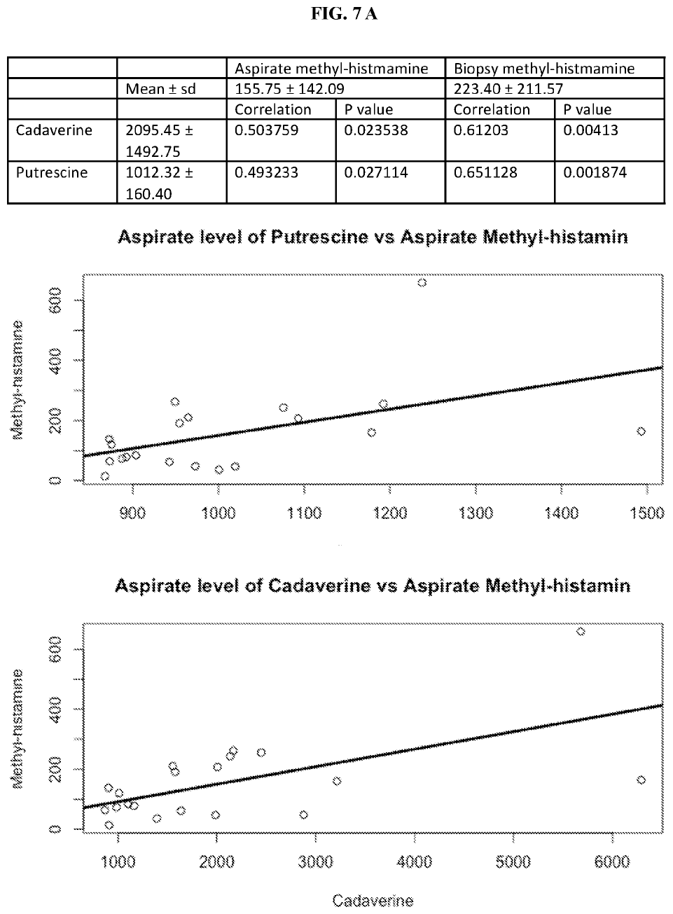

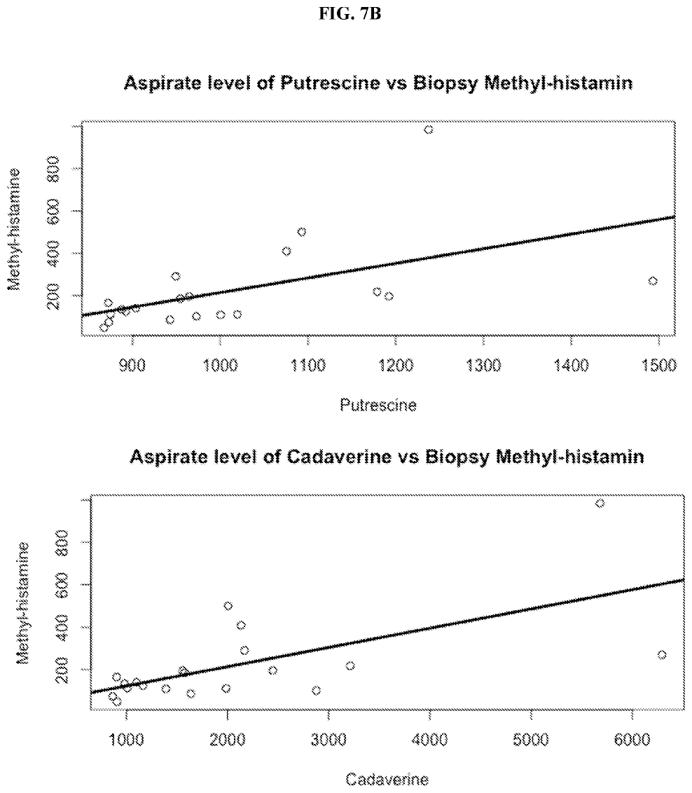

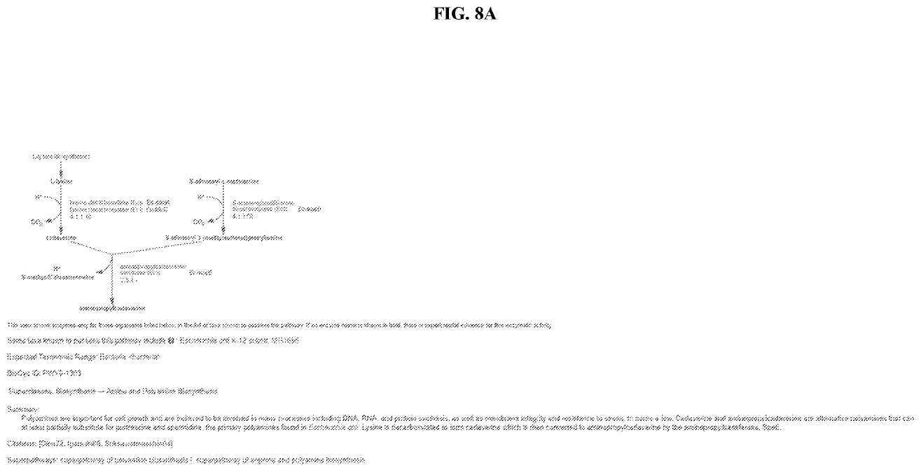

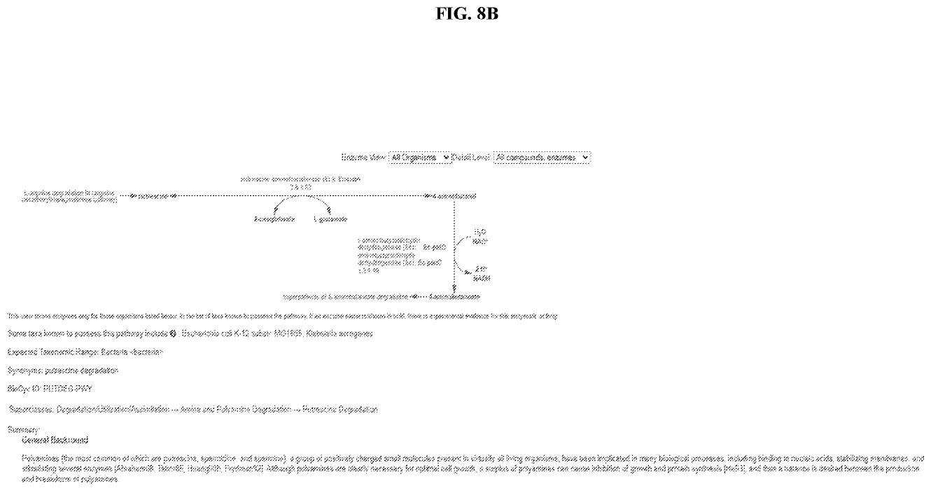

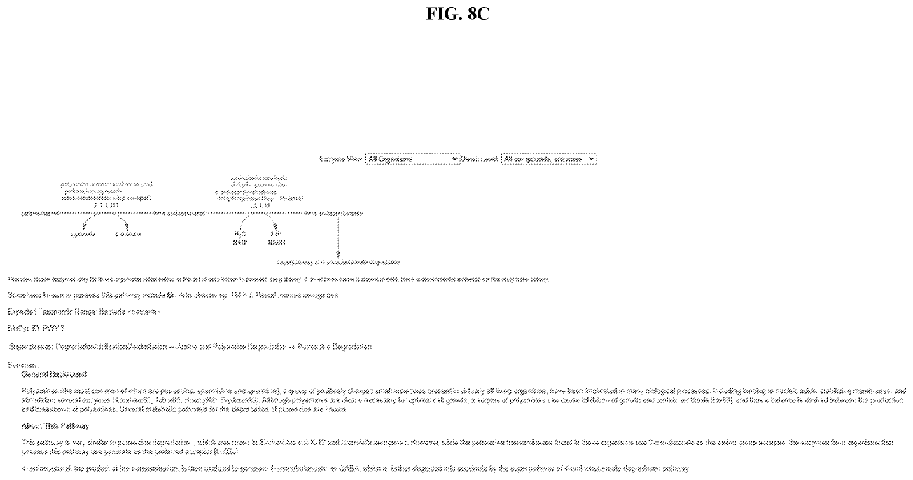

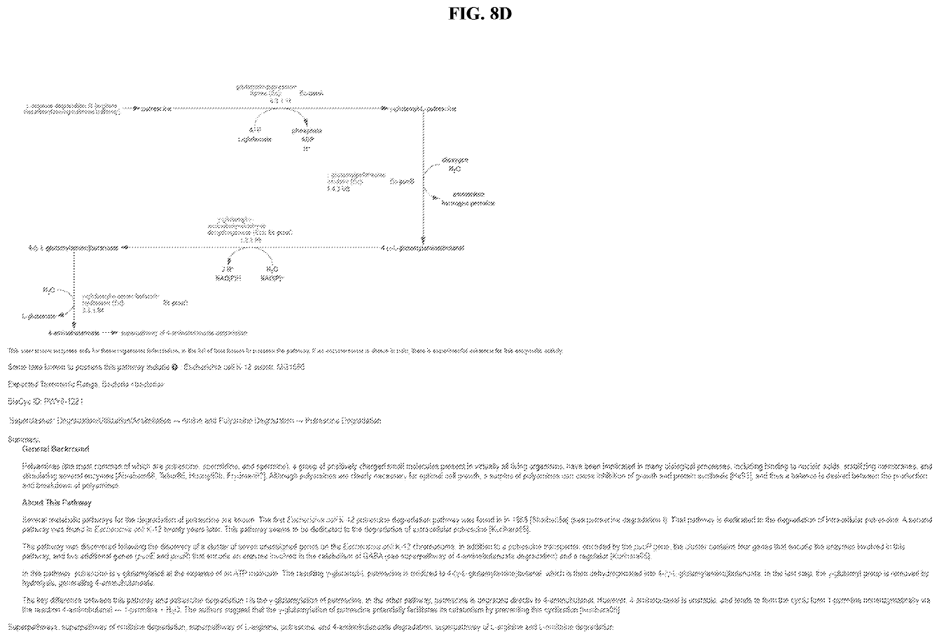

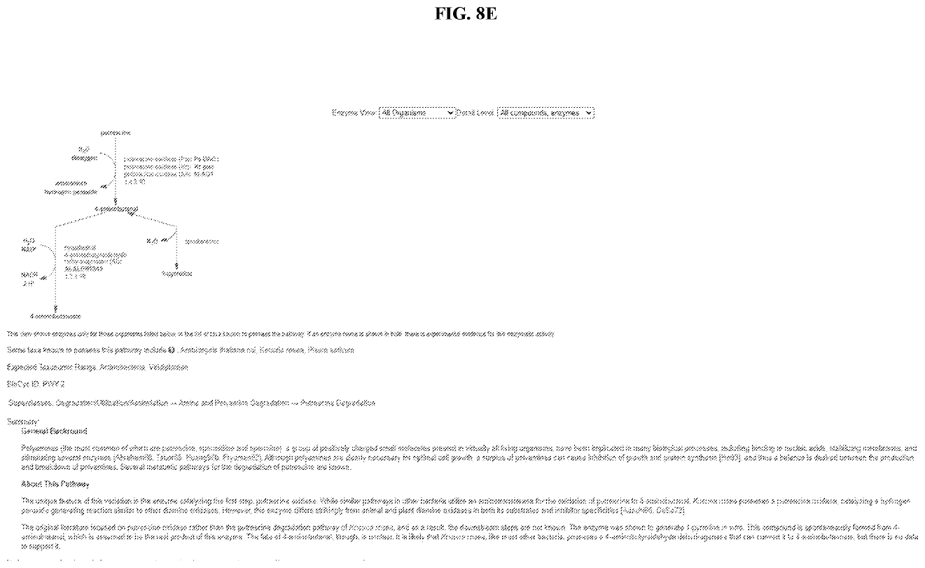

20 subjects undergoing EGD had small bowel aspiration under protected and sterile conditions along with duodenal biopsies. Of 20 subjects 5 had SIBO. Biogenic amines and their degradation products were measured using mass spectrometry. See FIG. 3A.

### Example 3

### Glossary

HILIC hydrophilic interaction liquid chromatography, a variant of normal-phase chromatography UHPLC ultra high-pressure liquid chromatography

ESI electrospray ionization. The method uses both negative ESI and positive ESI for negatively charged and positively charged molecules.

QTOF quadrupole time-of-flight mass spectrometer

MS/MS tandem mass spectrometry. After soft ionization by electrospray, the precursor (intact) charged molecules are fragmented by collision with gas atoms, usually Helium. Fragments are then analyzed by time-of-flight mass spectrometry to obtain accurate mass information at high resolution.

Resolving power also called resolution. In MS, resolving power defines the ability to distinguish co-eluting masses that have the same nominal mass, but different accurate mass.

MTBE methyl-tertiary butyl ether

MeOH methanol

BEH amide bridged ethylene hybrid amide column

QC quality control

IS, istd internal standard v/v volumetric ratio

InChI International Chemical Identifier key. Denotes the exact stereochemical and atomic description of chemicals and used as universal identifier in chemical databases.

rt retention time (minutes)

mz also m/z, or mass-to-charge ratio. In metabolomics, ions are almost exclusively detected as singly charged species.

rt_mz identifier for individual metabolites in the MassHunter Quantification method consisting of the retention time and the m/z value of specific compounds.

IUPAC International Union of Pure and Applied Chemists NIST National Institute of Standards and Technology PCA Principal Component Analysis

### Sample Extraction

Blood plasma or serum is best extracted following the protocols first published in Matyash V. et al., J. Lip. Res. 49 (2008) 1137-1146. One of the major differences to the earlier protocols by Folch or Bligh Dyer is that in the Matyash protocol, lipid extracts (labeled ‘org’ in the figure on the right) are separated from proteins and from polar hydrophilic small molecules (in the methanol/water phase, labeled ‘W’ in the figure on the right) in a way that the lipids are found in the top layer of liquid-liquid separations, rather than in the bottom layer. Decanting the top layer therefore ensures that extracts are not contaminated by proteins or polar compounds. The top layer is used for lipidomics while the bottom layer (methanol/water phase) is very suitable for HILIC-MS investigations. In some cases, other extraction methods may be used. For example, a mixture of acetonitrile/water/isopropanol (2:2:3) can be used to precipitate proteins and extract metabolites. In such cases, complex lipids would be found in HILIC-QTOF MS chromatograms in addition to small molecule metabolites such as betaine, choline and TMAO. Details of extraction methods for individual studies may vary and are available by inquiry.

### Data Acquisition

HILIC samples are most frequently injected using an Agilent 1290 UHPLC/Sciex TripleTOF 6600 mass spectrometer. Occasionally, HILIC samples may be injected using an Agilent 1290 UHPLC/Agilent 6550/6530 mass spectrometer or a Thermo Vanquish UHPLC/Thermo QExactive mass spectrometer. LC parameters are displayed in the table below; MS parameters vary by instrument and are available by request. The analytical UHPLC column is protected by a short guard column. This chromatography method yields excellent retention and separation of various small molecule metabolites with narrow peak widths of 2-5 s and very good within-series retention time reproducibility typically less than 1 s absolute deviation of retention times.

| Column | Waters Acquity Premier UPLC BEH Amide | |
|---|---|---|
| | Column (1.7 μm, 2.1 mm × 50 mm) | |
| Mobile | Ultrapure water with 10 mM ammonium | |
| phase A | formate + 0.125% formic acid, pH 3 | |
| Mobile | 95:5 v/v acetonitrile:ultrapure water w/ 10 mM | |
| phase B | ammonium formate + 0.125% formic acid, pH 3 | |
| Column | 45° | C. |
| temperature | | |
| Gradient | 0 min, 100% B; 0.5 min, 100% B; 1.95 min, 70% B; | |
| | 2.55 min, 30% B; 3.15 min, 100% B; 3.8 min, 100% B | |
| Flow rate | 0.8 | mL/min |
| Injection | 1-5 | μL |
| volume | | |

### Data Processing

Chromatograms first undergo a quality control check in which internal standards are examined for consistency of peak height and retention time. Raw data files are then processed using an updated version of MS-DIAL software which identifies and aligns peaks and then annotates peaks using both an in-house mzRT library and MS/MS spectral matching with NIST/MoNA libraries. All MS/MS annotations are then manually curated by a lab member to ensure that only high-quality compound identifications are included in the final report.

### Data Reporting

Data are reported including metadata. The ‘identifier’ column denotes the unique identifier for the technology platform, given as rt_mz. It is given for both identified and unidentified metabolites in the same manner. The ‘metabolite name’ column denotes the name of the metabolite, if the peak has been identified. A chemical name is not a unique identifier. We use names recognized by biologists instead of IUPAC nomenclature. The ‘InChI key’ column gives the unique chemical identifier defined by the IUPAC and NIST consortia. The ‘average rt’ and ‘average mz’ columns detail the retention time and mz values that were detected in a specific data processing sequence of chromatograms. These values may be slightly different from the values given in the ‘identifier column’. The ‘File ID’ row denotes the name of the raw data file.

The actual data are given as peak heights for the quantification ion (mz value) at the specific retention time (rt value). We provide peak heights instead of peak areas because peak heights are more precise for low abundant metabolites than peak areas, due to the larger influence of baseline determinations on areas compared to peak heights. Also, overlapping (co-eluting) ions or peaks are harder to deconvolute in terms of precise determinations of peak areas than peak heights.

Raw data peak heights need to be normalized to reduce the impact of within-series drifts of instrument sensitivity, caused by machine maintenance, aging and tuning parameters. There are many different types of normalizations in the scientific literature. We usually provide a variant of a ‘vector normalization’ in which we calculate the sum of all peak heights for all identified internal standards for each sample, denoted iTIC. Peak heights for all samples are then normalized to the total average sum of the internal standards (iTIC).

Equation used for normalizations of metabolite i of sample j:

metabolite ij,normalized=(metabolite ij,raw/iTIC j)*iTIC average

In some cases, such as when variable amounts of samples were extracted, the sum of all annotated metabolites (mTIC) may be used in place of the sum of the internal standards (iTIC). For studies exceeding ˜330 samples, SERRF normalization is used in place of iTIC normalization (for details, see Fan S, Kind T, Cajka T, et al. Systematic error removal using random forest for normalizing large-scale untargeted lipidomics data. Anal Chem. 2019; 91(5):3590-3596). Reported peak heights are ‘relative semi-quantifications’, meaning they are normalized peak heights. Because the average iTIC/mTIC will be different between series of analyses that are weeks or months apart (due to differences in machine sensitivity, tuning, maintenance status and other parameters), additional normalizations need to be performed if comparisons between disparate data sets are needed. In such cases, please contact our staff for more information regarding appropriate normalizations.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of”

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

1. A method of treating a biogenic amine related condition in a subject in need thereof, reducing one or more symptoms in a subject having a biogenic amine related condition, or decreasing biogenic amine level in a subject having a biogenic amine related condition, comprising administering to the subject a therapy selected from:
an elemental diet,
an enzyme to decrease production of an amine,
an enzyme to degrade an amine,
an antibiotic to decrease histamine producing microorganisms, putrescine producing microorganisms, cadaverine producing microorganisms, spermine producing microorganisms, or spermidine producing microorganisms in the gut,
a probiotic to decrease histamine producing microorganisms, putrescine producing microorganisms, cadaverine producing microorganisms, spermine producing microorganisms, or spermidine producing microorganisms in the gut, or
combinations thereof.

2. (canceled)

3. (canceled)

4. (canceled)

5. (canceled)

6. (canceled)

7. The method of claim 1, wherein the therapy is an elemental diet comprising low or absent precursor amino acids for microorganisms, wherein the precursor amino acid is histidine, arginine, lysine, ornithine, or combinations thereof.

8. The method of claim 1, wherein the therapy is an elemental diet and the elemental diet is selected from a semi or non-histidine diet, a semi or non-arginine diet, a semi or non-lysine/ornithine diet, or combinations thereof.

9. The method of claim 1, wherein the enzyme to decrease production of an amine comprises an amino acid decarboxylase to decrease production of histamine, putrescine, or cadaverine, or wherein the enzyme to decrease production of an amine comprises an amino acid decarboxylase to decrease production of spermidine or spermine.

10. (canceled)

11. The method of claim 9, wherein the amino acid decarboxylase is histidine decarboxylase, arginine decarboxylase, or lysine/ornithine decarboxylase, or wherein the amino acid decarboxylase is spermidine decarboxylase or spermine decarboxylase.

12. (canceled)

13. The method of claim 1,
wherein the enzyme inhibits amino acid decarboxylase and is selected from the group consisting of alpha-fluoromethylhistidine, alpha-Difluoromethyllysine (DEML), monofluoromethyllysine (MFML), alpha-Difluoromethylarginine, monofluoromethylagmatine, alpha-monofluoromethyl-3-4-dehydroarginine, alpha-Difluoromethylomithine (DFMO), agmatinase inhibitor, arginase inhibitor and combinations thereof, or
wherein the enzyme to degrade an amine comprises an aminotransferase, or
wherein the enzyme to degrade an amine comprises a monoamine oxidase (MAO), diamine oxidase (DAO), or both, or
wherein the enzyme to degrade an amine is histamine N-methyltransferase (HNMT), or
wherein the enzyme to degrade an amine is selected from the group consisting of gamma-glutamylcadaverine synthetase, gamma-glutamylputrescine synthetase, gamma-L-glutamylputrescine synthetase, gamma-glutamylcadaverine oxidoreductase, gamma-glutamylputrescine oxidoreductase, or combinations thereof, or
wherein the enzyme to degrade an amine is selected from the lysine decarboxylase, aminopropylcadverine synthase, S-adenosylmethionine decarboxylase, putrescine aminotransferase, gamma-aminobutyraldehyde dehydrogenase, aminobutyraldehyde dehydrogenase, polyamine aminotransferase, putrescine-pyruvate aminotransferase, aminobutyraldehyde dehydrogenase, 4-aminobutyraldehyde dehydrogenase, glutamate-putrescine ligase, gamma-glutamylputrescine oxidase, gamma-glutamyl-gamma-aminobutraldehyde dehydrogenase, gamma-glutamyl-gamma-aminobutyrate hydrolase, putrescine oxidase, 4-aminobutyradldehyde dehydrogenase, or combinations thereof.

14. (canceled)

15. The method of claim 13, wherein the aminotransferase is putrescine aminotransferase, cadaverine aminotransferase, or both.

16. (canceled)

17. (canceled)

18. (canceled)

19. (canceled)

20. The method of claim 1, wherein the enzyme to degrade an amine comprises diamine oxidase (DAO), and further comprise an enzyme that degrade histamine, putrescine, cadaverine, spermidine, spermine or combinations thereof.

21. The method of a claim 1, wherein the enzyme is enteric coated.

22. The method of claim 1, wherein the antibiotic to decrease histamine producing microorganisms, putrescine producing microorganisms, or cadaverine producing microorganisms in the gut is an antibiotic effective against gram-negative bacteria, or wherein the antibiotic to decrease spermidine producing microorganisms, or spermine producing microorganisms in the gut is an antibiotic effective against gram-negative bacteria.

23. (canceled)

24. The method of claim 1, wherein the antibiotic comprises rifaximin, metronidazole or both.

25. The method of claim 1, wherein the antibiotic is administered in combination with N-acetylcysteine (NAC).

26. The method of claim 1, wherein the probiotic comprises histamine metabolizing bacterium, putrascine metabolizing bacterium, cadaverine metabolizing bacterium, spermidine metabolizing bacterium, spermine metabolizing bacterium, or a combination thereof.

27. The method of claim 1, wherein the therapy further comprises amine-negative or amine oxidizing bacteria, sodium sorbate, citric acid, succinic acid, malic acid, D-sorbitol, thymol, lemongrass, turmeric, capsaicin, piperine or combinations thereof.

28. The method of claim 1, wherein the biogenic amine related condition is functional dyspepsia, irritable bowel syndrome (IBS), asthma, migraine, food allergies, environmental allergies, urticaria, mast cell activation syndrome, hereditary alpha tryptasemia, halitosis, or combinations thereof.

29. The method of claim 1, wherein the subject has been detected to have a quantity of one or more of the following microorganisms higher than each microorganism's reference value: Raoultella ornithinolytica, Morganella morganii, Entercoccus faecalis, Proteus mirabilis, Pseudomonas spp., Klebsiella oxytoca, K. aerogenes, K. pneumoniae, Staphylococcus aureus, Stenotrophomonas maltophilia, Escherichia coli, or Enterobacter cloacae, Campylobacter jejuni, Streptococcus pneumoniae, Bacillus subtilis, Archaea, Actinobacteria spp., Firmicutes spp., Proteobacteria spp., or Bacteroidetes spp.

30. The method of claim 1, wherein the subject has been detected to have a quantity of one or more of the following microorganisms higher than each microorganism's reference value: Raoultella ornithinolytica, Morganella morganii, Entercoccus faecalis, Proteus mirabilis, Pseudomonas spp., Klebsiella oxytoca, K. aerogenes, K. pneumoniae, Staphylococcus aureus, Stenotrophomonas maltophilia, Escherichia coli, or Enterobacter cloacae.

31. The method of claim 1, wherein the subject has been detected to have a quantity of one or more of the following microorganisms higher than each microorganism's reference value: Campylobacter jejuni, Streptococcus pneumoniae, Bacillus subtilis, Archaea, S. maltophilia, E. coli, Pseudomonas spp., Actinobacteria spp., Firmicutes spp., Proteobacteria spp., or Bacteroidetes spp.

32. A method of selecting a therapy for a biogenic amine related condition for a subject, comprising:
detecting a quantity of one or more of the following microorganisms: Raoultella ornithinolytica, Morganella morganii, Entercoccus faecalis, Proteus mirabilis, Pseudomonas spp., Klebsiella oxytoca, K. aerogenes, K. pneumoniae, Staphylococcus aureus, Stenotrophomonas maltophilia, Escherichia coli, or Enterobacter cloacae, Campylobacter jejuni, Streptococcus pneumoniae, Bacillus subtilis, Archaea, Actinobacteria spp., Firmicutes spp., Proteobacteria spp., or Bacteroidetes spp.; and
selecting a therapy for the subject when the quantity of one or more of the microorganisms are each higher than its reference value,
wherein the therapy is selected from:
an elemental diet,
an enzyme to decrease production of an amine,
an enzyme to degrade an amine,
an antibiotic to decrease histamine producing microorganisms, putrescine producing microorganisms, cadaverine producing microorganisms, spermidine producing microorganisms, or spermine producing microorganisms in the gut,
a probiotic to decrease histamine producing microorganisms, putrescine producing microorganisms, cadaverine producing microorganisms, spermidine producing microorganisms, or spermine producing microorganisms in the gut, or
combinations thereof.

33. The method of claim 32, wherein detecting comprises detecting a quantity of one or more of the following microorganisms: Raoultella ornithinolytica, Morganella morganii, Entercoccus faecalis, Proteus mirabilis, Pseudomonas spp., Klebsiella oxytoca, K. aerogenes, K. pneumoniae, Staphylococcus aureus, Stenotrophomonas maltophilia, Escherichia coli, or Enterobacter cloacae, or
wherein detecting comprises detecting a quantity of one or more of the following microorganisms: detecting a quantity of one or more of the following microorganisms: Campylobacter jejuni, Streptococcus pneumoniae, Bacillus subtilis, Archaea, S. maltophilia, E. coli, Pseudomonas spp., Actinobacteria spp., Firmicutes spp., Proteobacteria spp., or Bacteroidetes spp.

34. (canceled)

35. The method of claim 32, wherein the detection is made in a biological sample obtained from the subject, wherein the biological sample comprises small bowel aspirate, gut mucosal biopsies, blood, breath, or a combination thereof.

36. (canceled)

37. The method of claim 32, further comprising administering the selected therapy.

38. A method of quantifying a microorganism in a biological sample from a subject comprising:
assaying the biological sample for one or more of the following microorganisms: Raoultella ornithinolytica, Morganella morganii, Entercoccus faecalis, Proteus mirabilis, Pseudomonas spp., Klebsiella oxytoca, K. aerogenes, K. pneumoniae, Staphylococcus aureus, Stenotrophomonas maltophilia, Escherichia coli, or Enterobacter cloacae, Campylobacter jejuni, Streptococcus pneumoniae, Bacillus subtilis, Archaea, Actinobacteria spp., Firmicutes spp., Proteobacteria spp., or Bacteroidetes spp.; and
detecting the quantity of the one or more microorganisms,
wherein the subject has a biogenic amine related condition or exhibit a symptom of the biogenic amine related condition.

39. The method of claim 38, wherein assaying comprises assaying the biological sample for one or more of the following microorganisms: Raoultella ornithinolytica, Morganella morganii, Entercoccus faecalis, Proteus mirabilis, Pseudomonas spp., Klebsiella oxytoca, K. aerogenes, K. pneumoniae, Staphylococcus aureus, Stenotrophomonas maltophilia, Escherichia coli, or Enterobacter cloacae, or
wherein assaying comprises assaying the biological sample for one or more of the following microorganisms: Campylobacter jejuni, Streptococcus pneumoniae, Bacillus subtilis, Archaea, S. maltophilia, E. coli, Pseudomonas spp., Actinobacteria spp., Firmicutes spp., Proteobacteria spp., or Bacteroidetes spp.

40. (canceled)

41. The method of claim 38, further comprising comparing the quantity of the one or more microorganisms to each microorganism's reference value.
